(19) United States
(12) Patent Application Publication
(10) Pub. No.: US 2019/0015245 A1
(43) Pub. Date: Jan. 17, 2019

(54) DEVICE FOR TREATMENT OF SLEEP DISORDERS

(71) Applicant: NIDRA MEDICAL TECHNOLOGIES, INC., Cary, NC (US)

(72) Inventor: Hrushikesh Vaidya, Thane (IN)

(21) Appl. No.: 16/065,411

(22) PCT Filed: Dec. 26, 2016
(86) PCT No.: PCT/IB2016/058005

§ 371 Date: Jun. 22, 2018

(30) Foreign Application Priority Data
Dec. 29, 2015 (IN) 4911/MUM/2015

(51) Int. Cl. A61F 5/56

(52) U.S. Cl. CPC A61F 5/56; A61F 5/05891

(57) ABSTRACT

Method and devices for aiding in preventing obstructive sleep apnea are disclosed. In one aspect a device for aiding in preventing of obstructive sleep apnea is disclosed. The device may include a chin attachment member including a coupling to couple to an underside of a chin of a patient, a jaw support shaped to engage a jaw of the patient and having at least one locking member, and a body support shaped to rest on a chest of the patient. The coupling may an adhesive surface of the chin attachment member. Some embodiments may include a connection member, the connection member coupling the jaw support to the body support. The connection member may be a spring. In some embodiments, the connection member may be rigid.

### TECHNICAL FIELD

The present disclosure generally relates to the field of devices, systems, and methods for treating sleep disorders and, more particularly, to an externally applied device for the treatment of obstructive sleep apnea.

### BACKGROUND

Obstructive sleep apnea hypopnea syndrome (OSAHS or simply, obstructive sleep apnea) remain a common and serious medical disorder affecting 2-4% of middle aged women and men in the United States in spite of many medical and surgical advances in its treatment. The condition is characterized by recurrent sleep induced collapse of the pharyngeal airway, which leads to hypoxaemia and hypercapnia with arousal from sleep being required to re-establish airway patency.

Obstructive sleep apnea decreases oxygen levels in the body resulting in daytime fatigue, cardiovascular problems and, in some cases, may even result in death. It has been suggested that patients with obstructive sleep apnea have anatomical predisposition to airway collapse. It has been observed that the protective mechanisms that maintain the patency of the pharyngeal airway by increasing activity of pharyngeal dilator muscles, fails during sleep resulting in the subsequent collapse of the pharyngeal airway behind the palate or tongue or both. When the pharyngeal collapses, it is generally in the velopharynx (behind the soft palate) or oropharynx (from the top soft palate to the epiglottis) or both.

Therapies used in the art to treat obstructive sleep apnea include straps, dental devices, continuous positive airway pressure, and surgical procedures. However, despite many such advances, patients find these devices ineffective, inconvenient, or uncomfortable and patient compliance is a problem. Therefore, there still remains a need for treatment of obstructive sleep apnea with improved effectiveness, patient compliance, and convenience.

### SUMMARY

The present disclosure is directed to improved and convenient to use systems, devices and methods for preventing obstructive sleep apnea.

A device for aiding in preventing of obstructive sleep apnea is disclosed. The device may include a chin attachment member including a coupling to couple to an underside of a chin of a patient, a jaw support shaped to engage a jaw of the patient and having at least one locking member, and a body support shaped to rest on a chest of the patient. The coupling may an adhesive surface of the chin attachment member. Some embodiments may include a connection member, the connection member coupling the jaw support to the body support. The connection member may be a spring. In some embodiments, the connection member may be rigid.

Some embodiments may include a tensioning member having an elongated body extending between a first end and a second end, the first end configured to couple to the chin attachment member, and at least one locking member may be configured to receive the second end of the tensioning member. In some embodiments, the locking member is configured to engage the tensioning member. In some embodiments, the jaw support and locking member are configured to hold the tension member in tension with the attachment member and apply a force the anterior direction to the tissue of the sub-mandibular region of a patient. The tensioning member may include an adjustment feature in the form selected from teeth, threads, a rough surface for friction locking, and adjustment apertures. the elongated body of the tensioning member may include polyamide, polypropylene, polycarbonate, ABS, nylon, polyester, or silk. The jaw support and locking member may be configured to hold the tension member in tension without protraction of a mandible of the patient.

A method for treating obstructive sleep apnea is disclosed. The method may include attaching an attachment member to a patient at a location under the chin of the patient, coupling a jaw assembly to the patient, the jaw assembly being coupled such that it is held about the mental and buccal portions of the patient's face, applying a force the tissue of the sub-mandibular region of the patient in an anterior direction. The method may also include maintaining airway patency while the patient is sleeping or under treatment of anesthesia. In some embodiments, the method may include coupling the attachment member to the jaw assembly with a tensioning member.

In some embodiments, the tensioning member may be an elongated body having a first end and a second end, and may be coupled to the attachment member at a first end that may be received though a locking member of the jaw assembly. Some embodiments may include engaging the elongated body of the tensioning member by the locking member to hold the tensioning member in tension between the locking member and the attachment member. In some embodiments, the tensioning member transmits the force from the jaw assembly though the attachment member to the tissue of the sub-mandibular region of the patient. The airway patency may be maintained without protraction of a mandible of the patient. Some embodiments may include providing a body assembly coupled to the jaw assembly, and placing the body support on the upper chest of the patient. Some embodiments may also include providing a body assembly coupled to the jaw assembly via connecting members slidingly engaged with the jaw assembly at a coupling and adjusting the body assembly position relative to the jaw assembly by sliding the connecting members in the coupling.

These and other features, aspects and advantages of the present disclosure will become better understood with reference to the following drawings, description and claims.

### BRIEF DESCRIPTION OF THE DRAWINGS

FIG. 1 illustrates a perspective view of a neck assembly according to one or more embodiments disclosed herein.

FIG. 2 depicts a front view of the neck assembly of FIG. 1 according to one or more embodiments disclosed herein.

FIG. 3 depicts a rear view of the neck assembly of FIG. 1 according to one or more embodiments disclosed herein.

FIG. 4 depicts a top view of the neck assembly of FIG. 1 according to one or more embodiments disclosed herein.

FIG. 5 illustrates perspective view of a chin attachment member applied under the chin of a user according to one or more embodiments disclosed herein.

FIG. 6 illustrates a side view of a user wearing a neck collar assembly according to one or more embodiments disclosed herein.

FIG. 7 illustrates a side view of a user wearing a neck collar assembly with the collar assembly in cross section according to one or more embodiments disclosed herein.

FIG. 8 illustrates a side view of a user wearing a neck collar assembly with applied forces shown according to one or more embodiments disclosed herein.

FIG. 9 illustrates a perspective view of a tensioning member according to one or more embodiments disclosed herein.

FIG. 10 illustrates a perspective view of a tensioning member attached to a chin attachment member according to one or more embodiments disclosed herein.

FIG. 11 illustrates another perspective view of a tensioning member attached to a chin attachment member according to one or more embodiments disclosed herein.

FIG. 12 illustrates yet another perspective view of a tensioning member attached to a chin attachment member according to one or more embodiments disclosed herein.

FIG. 13 illustrates variant perspective view of a tensioning member attached to a chin attachment member according to one or more embodiments disclosed herein.

FIG. 14 illustrates another variant perspective view of a tensioning member attached to a chin attachment member according to one or more embodiments disclosed herein.

FIG. 15 illustrates yet another variant perspective view of a tensioning member attached to a chin attachment member according to one or more embodiments disclosed herein.

FIG. 16 illustrates perspective view of a chin attachment member according to one or more embodiments disclosed herein.

FIG. 17 illustrates perspective view of a tensioning member in the form of an elongated body according to one or more embodiments disclosed herein.

FIG. 18 illustrates variant perspective view of a tensioning member in the form of a string according to one or more embodiments disclosed herein.

FIG. 19 illustrates variant perspective view of a tensioning member in the form of a mechanical clamp according to one or more embodiments disclosed herein.

FIG. 20 illustrates variant perspective view of a tensioning member in the form of a suction cup according to one or more embodiments disclosed herein.

FIG. 21 illustrates perspective view of a tensioning member with adjustment feature according to one or more embodiments disclosed herein.

FIG. 22 illustrates variant perspective view of a tensioning member with adjustment feature according to one or more embodiments disclosed herein.

FIG. 23 illustrates a neck collar assembly according to one or more embodiments disclosed herein.

FIG. 24 illustrates a neck collar assembly according to one or more embodiments disclosed herein.

FIG. 25 illustrates a neck collar assembly according to one or more embodiments disclosed herein.

FIG. 26 illustrates a neck assembly according to one or more embodiments disclosed herein.

FIG. 27 illustrates a suction system according to one or more embodiments disclosed herein.

FIG. 28 illustrates a suction assembly according to one or more embodiments disclosed herein.

FIG. 29 illustrates a suction assembly and neck collar assembly according to one or more embodiments disclosed herein.

FIG. 30 illustrates a suction assembly according to one or more embodiments disclosed herein.

FIG. 31 illustrates variant perspective view of a tensioning member according to one or more embodiments disclosed herein.

FIG. 32 illustrates a neck collar assembly according to one or more embodiments disclosed herein.

FIG. 33 illustrates a neck collar assembly on a patient according to one or more embodiments disclosed herein.

Similar reference characters denote corresponding features consistently throughout the attached drawings.

### DETAILED DESCRIPTION

Various features are described below that can each be used independently of one another or in combination with other features. Broadly, embodiments of the present disclosure generally provide a neck assembly configured to prevent obstructive sleep apnea.

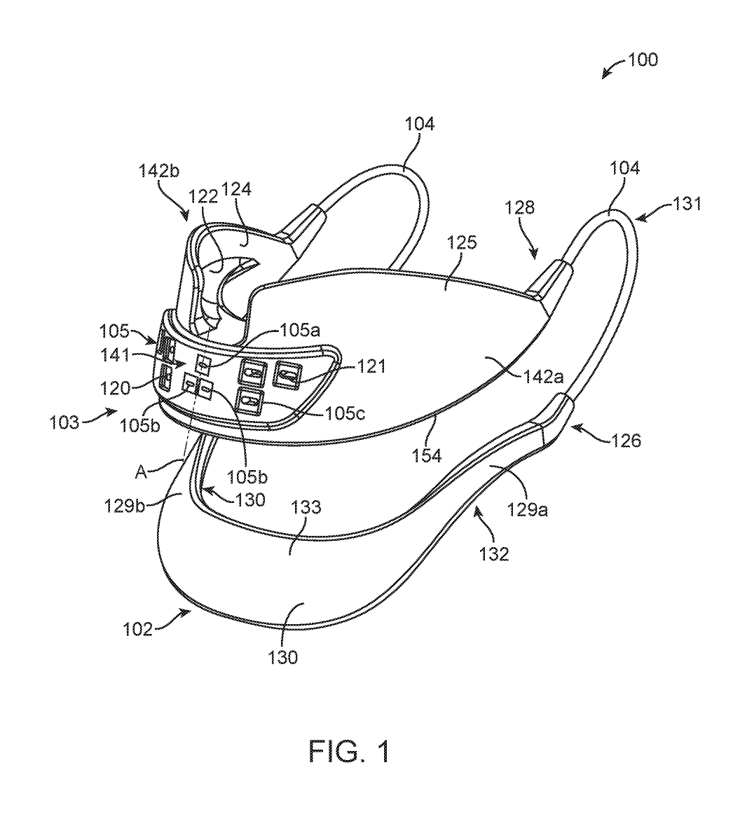

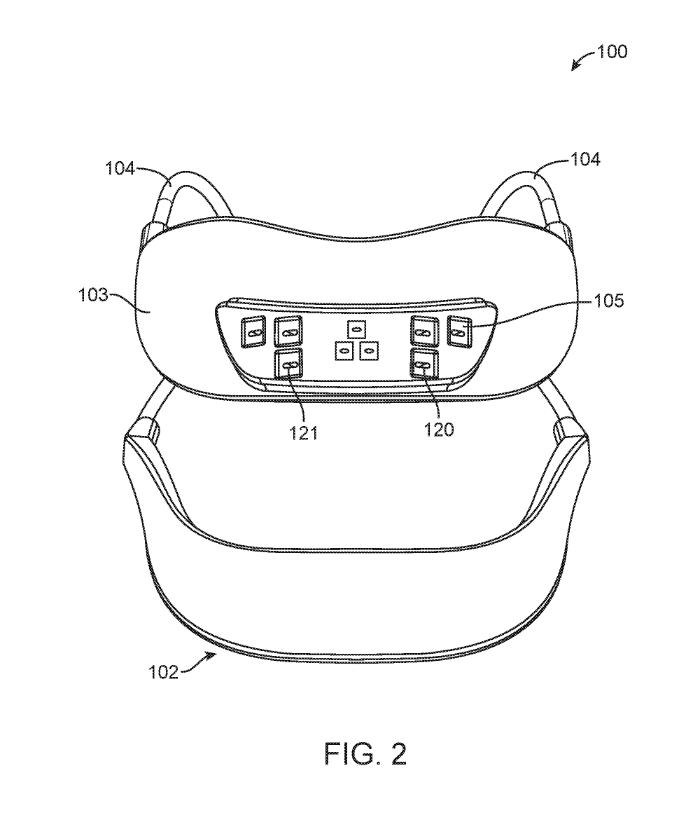

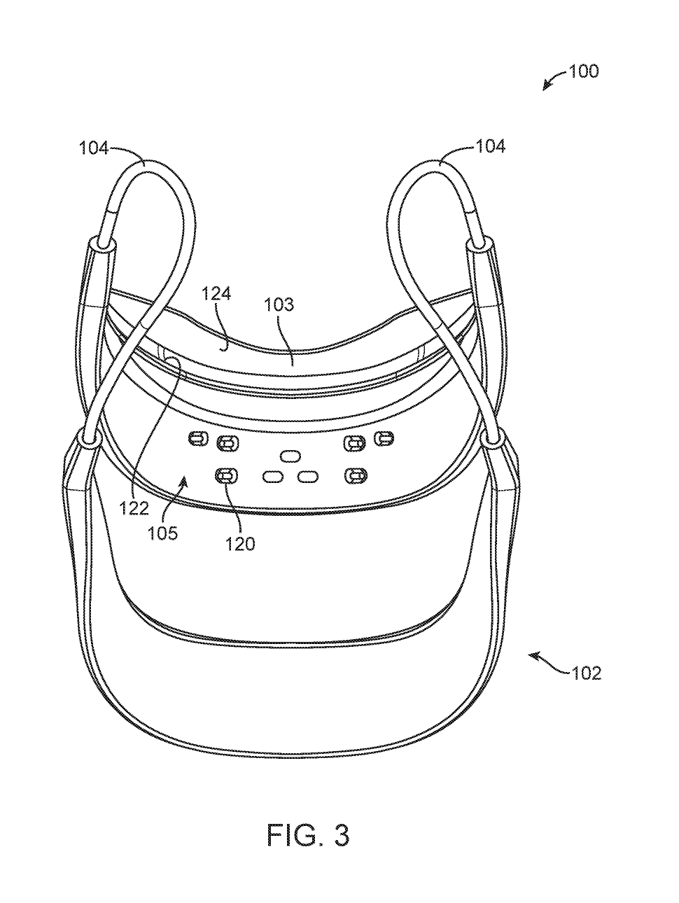

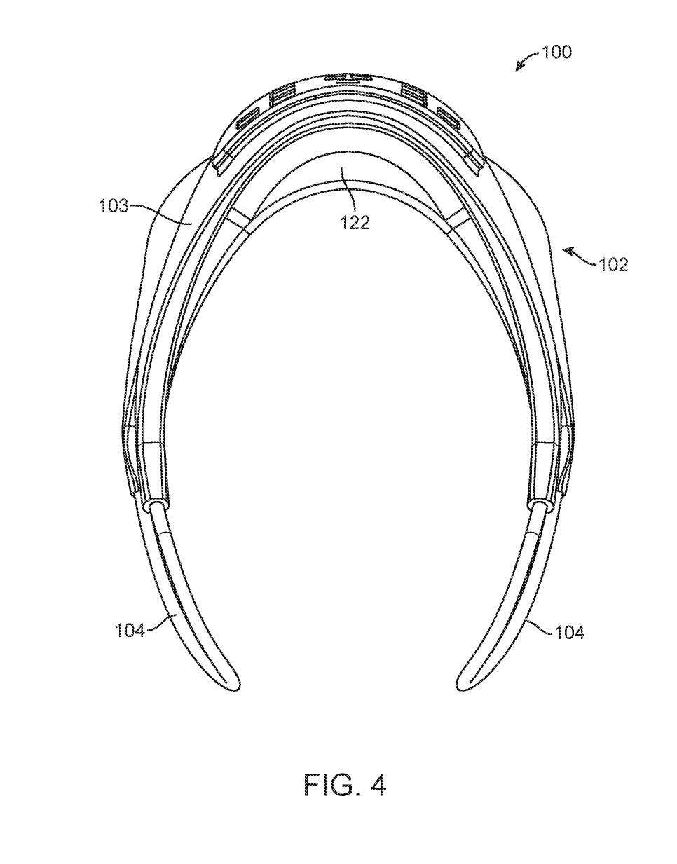

Referring to FIGS. 1-4, a neck assembly 100 is shown in perspective, front, bottom, and top views, respectively. The neck assembly 100 may include a body support 102 connected to a jaw support 103 via a connecting member 104.

The body support 102 includes an elongated body extending between two ends. Arms 129a, 129b extend from each of the two ends of the body of the body support 102. A coupling 126 is located at distal end of each of the arms 129a, 129b and couples the body support 102 to a respective connecting member 104. The elongated body includes a patient facing surface 130 that rests on the body of the patient and an opposite, outward facing surface 133, that faces away from the patient's body. In some embedment's, the body support 102 may rest on the chest of a patient and the patient facing surface 130 may be shaped to correspond to the shape of the outer surface of the patient in the upper chest area. For example, in some embodiments the patient facing surface 130 may be configured to comfortably rest on the patient's body at or near the collar bones by having a patient facing surface 130 that is shaped to account for the protrusions of a patient's collar bones. For example, the extensions 129a, 129b include a curved portion 132 to account for the collar bones of a patient.

The connecting member 104 is coupled to the body support 102 at a first end via coupling 126 and is coupled to the jaw support 103 at a second end via coupling 128. In some embodiments, the connecting member 104 may be releasably coupled to one or both of the body support 102 and the jaw support 103. Releasably coupling the connecting member 104 allows for customization of neck assembly for different sized and shaped patients.

In some embodiments, a plurality of connecting members 104 may be provided to a doctor to aid in fitting the neck assembly 100 to the patient. For example, a longer connecting member 104 may be used with patients that have longer necks and shorter connecting members 104 may be used with patients having shorter necks. In some embodiments, the different connecting members 104 may have bends 131 with different a radius and arc length so as to provide options for fitting the neck assembly 100 to a patient.

In some embodiments, the connecting member 104 may be fixedly coupled to one or more of the body support 102 and next support 103. In some embodiments, the connecting member 104 may be integrally formed with one or more of the body support 102 and neck support 103.

In some embodiments, the connecting member 104 may be rigid while in other embodiments, the connecting member 104 may be flexible.

As discussed above, the jaw support 103 is coupled to the connecting member 104. The jaw support 103 is configured to engage with the jaw and a surface of the underside of a patient's chin to apply an anterior force to the skin under the jaw. This anterior force is transferred though the submandibular tissue and results in tensioning of the genioglossus and geniohyoid muscles which maintains the patient's airway and aids in preventing obstructive sleep apnea. In some embodiments, the force is transferred to the hyoid bone to pull the hyoid bone anteriorly to increase the opening of the airway. This aspect may be useful to prevent severe obstructive sleep apnea.

The jaw support 103 may be a curved structure having a central portion 141, also called the mental portion, that is shaped to fit with a patient's chin, e.g., the mental region of the patient's face. Two arms 142a, 142b extend from the central portion 141 of the jaw support. The two arms 142a, 142b are sharped to fit with one or more of the buccal, parotid, and submaxillary portions of a patient's face. The central portion 141, and the two arms 142a, 142b may form a substantially U-shaped structure or semicircular structure that fits the shape of the patient's chin and jaw.

The body of the jaw support 103 includes a first, inward or patient facing surface 124, that is configured to face the patient and a second, outward facing surface 125, which faces away from the body of the patient Like the patient facing surface 130 of the body support 102, the patient facing surface 124 may be shaped to correspond to the shape of the outer body of the patient's face, for example the patient's chin and jaw. In some embodiments, the patient facing surface 124 may be configured to comfortably rest on the patient's body at or near one or more of the mental, buccal, parotid, and submaxillary regions of the patient's face.

As seen in FIGS. 1, 3, and 4, a shelf 122 may extend from the patient facing surface 124 of the jaw support 103. In some embodiments, the shelf 122 extends from a central portion 141 of the jaw support 103 and may have a shape that corresponds to a shape of the underside of patient's mental or chin region. In some embodiments, the shelf 122 extends from the one or more of the arms 142a, 142b and may have a shape that corresponds to a shape of the underside of a patient's jaw, in particular the submaxillary region of the patient's face.

In some embodiments, the shelf may extend from the patient facing surface 124 along the first of the arms 142a, through the central portion of the 141, and the second of the arms 142b. In some embodiments, the jaw support may include multiple shelves 122 or discrete shelf portions. For example, the jaw support may include a first shelf that extends from the inner surface 124 at the central portion 141 of the jaw support 103, and second and third shelves that extend from the inner surface 124 of each of the arms 142a, 142b, respectively.

The jaw support 103 may include a plurality of couplings, such as locking members 105, to allow for passage and retention of a tensioning member 106. The locking member 105 may include an aperture 120 through the body of the jaw support 103. The aperture 120 allows for passage of the tensioning member 106 through the jaw support 103 and permits a user, such as a patient, to pull on and tension the tensioning member 106.

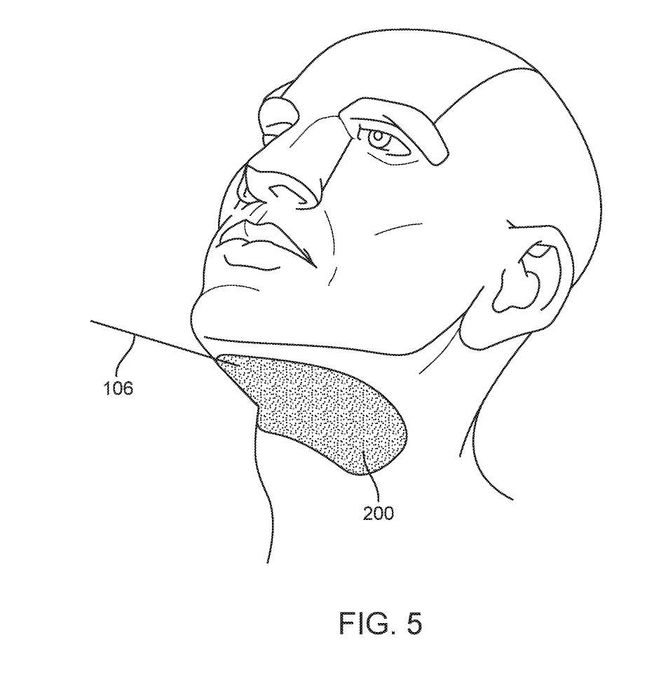

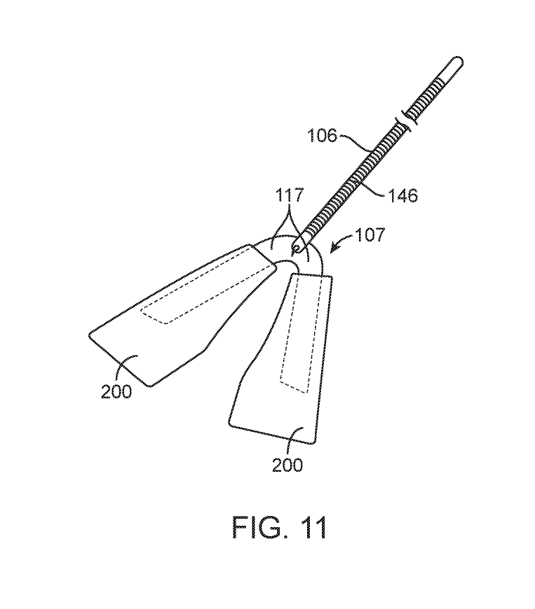

The locking member 105 also includes a means for retaining the tensioning member 106 (see e.g., FIGS. 5-7). For example, a locking member 105 may include a pawl within or proximate to the aperture that engages with and adjustment feature, such as teeth on the tensioning member 106 to form a ratchet coupling that allows movement of the tensioning member 106 though the locking member 105 and aperture 120 in a first direction and resists movement of the tensioning member in a second direction, which may be opposite the first direction. Such embodiments may allow the user to tension the tensioning member by pulling on it in a first direction and then maintain tension in the tensioning member 106 when the pawl 121 engages with teeth of the tensioning member, see for example, the teeth 146 of the tensioning member 106 in FIG. 11. In some embodiments, the adjustment feature can be in the form of a ratchet, threaded screw, rough surface for friction locking, adjustment holes or other arrangement.

The tensioning member 106 is attached to an attachment member 200 (see FIG. 5) which may be applied under the chin of the user using adhesive, as described above, vacuum suction, or mechanical clamping. The attachment member 200 transfers the force from the tensioning member 106 to the patient.

Referring back to FIGS. 1-4, in some embodiments, the jaw support 103 may include a plurality of couplings, such as locking members 106, arranged in an array on the central portion 141 of the jaw support 103. In some embodiments, the locking members 106 may be aligned with a centerline A of the jaw support 103. The centerline A of the jaw support 103 is located along the center of the jaw support 103 along a line that is equidistant between the ends of the jaw support, for example between the ends of the arms 142a, 142b or between the couplings 128 of each arm 142a, 142b. In some embodiments, the center line A extends parallel to the longitudinal (vertical) axis of a patient when the patient is wearing the jaw support 103.

In some embodiments, the locking member a located proximate the center line A. For example, the locking members 105b are located a distance of less than 0.5 cm on either side of the center line A. In some embodiments, the locking members are located less than 1 cm, less than 2 cm, or less than 3 cm from the center line A.

In some embodiments, the locking members 105 are located above the shelf 122, below the shelf 122, or a first one or more of the locking members 105 are located above the shelf 122 and a second one or more of the locking members 105 are located below the shelf 122.

In some embodiments, the neck assembly 100 can be flexible structure wherein the jaw support 103 and the body support 102 can be connected by means of a flexible connecting member 104 in the form of a spring.

The neck assembly 100 or one or more of the body of the jaw support 103, the connecting member 104, and the body of the body support 102 can be made in a single layer construction from materials including but not limited to polyurethane foam, EVA foam, or similar soft materials adaptable to provide both rigid support and comfort while wearing.

The neck assembly 100 can be made from more than one layer. The external layer which may include the exterior facing surface 125 may be made from rigid material including, but not limited to, fiber reinforced plastic, ABS, polycarbonate, polypropylene or similar engineering polymer or metal including but not limited to stainless steel and aluminum. The internal layer which may include the patient facing surface 124 that may be in contact with the user's skin can be made of soft materials including but not limited to foam, silicone, silicone gel or similar material adaptable to provide comfort while wearing.

The jaw support 103 may be rigidly connected to the body support 102 using the connecting member 104. The body support 102 is configured to support and stabilize the neck assembly 100 and adapted to be in contact with the part of user's body. While in use, the body support 102 aids in transmitting the forces created by the tensioning member 106 to the user's chest and torso.

In one embodiment, the connecting member 104 can be in the form of a flexible member, preferably a spring. The spring may allow movement of the user's neck while wearing the collar 101. In another embodiment, the connecting member 104 can be in the form of a belt designed to hold the jaw support 103 and the body support 102 together as a unit to allow movement of the neck.

With reference to FIG. 5, force transmitting means, such as a tensioning member 106 and a means for coupling the tensioning member 106 to a patient are shown. The means for coupling the tensioning member 106 to a patient may include an attachment member 200, such as an adhesive sheet, are shown in use on a patient. As shown, the attachment member 200 may be configured to be attached to the underside of the chin and jaw of a patient. In particular, the attachment member 200 may be sized and shaped to be coupled to the patient at one or more of the submental, submaxillary, hyoid, subhyoid, and carotid fossa of the face and neck of a patient. In some embodiments, the attachment member 200 may be sized and shaped such that it transfers a force from the tensioning member 106 to the geniohyoid muscle and genioglossus muscle such that the sub-mandibular tissue is moved anteriorly to maintain the tone of the genioglossus and geniohyoid muscles and thereby maintaining airway patency while sleeping or during administration of anesthesia.

The tensioning member 106 may be attached or otherwise coupled to the attachment member 200. In some embodiments the tensioning member may be integral with the attachment member 200.

The tensioning member 106 can be made from materials including but not limited to polyamide, polypropylene, polycarbonate, ABS or such engineering plastics or in string form. It can preferably be made from materials including nylon, polyester, silk or similar high strength fibers.

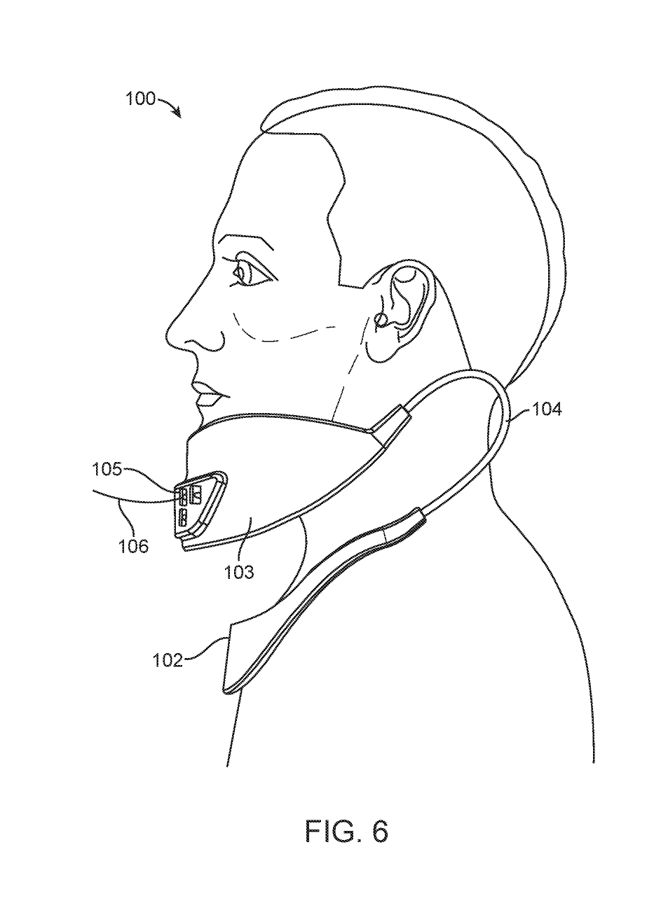

With reference to FIGS. 6-8, during use, the neck assembly 100 is comfortably placed around the neck of the user and configured to hold the jaw support 103 to engage jaw of the user. For example, the jaw may rest against or otherwise engage with the patient facing surface 124 of the body of the jaw assembly 103 and the upper surface 133 of the shelf 122. The body support 102 rests against the chest of the patient.

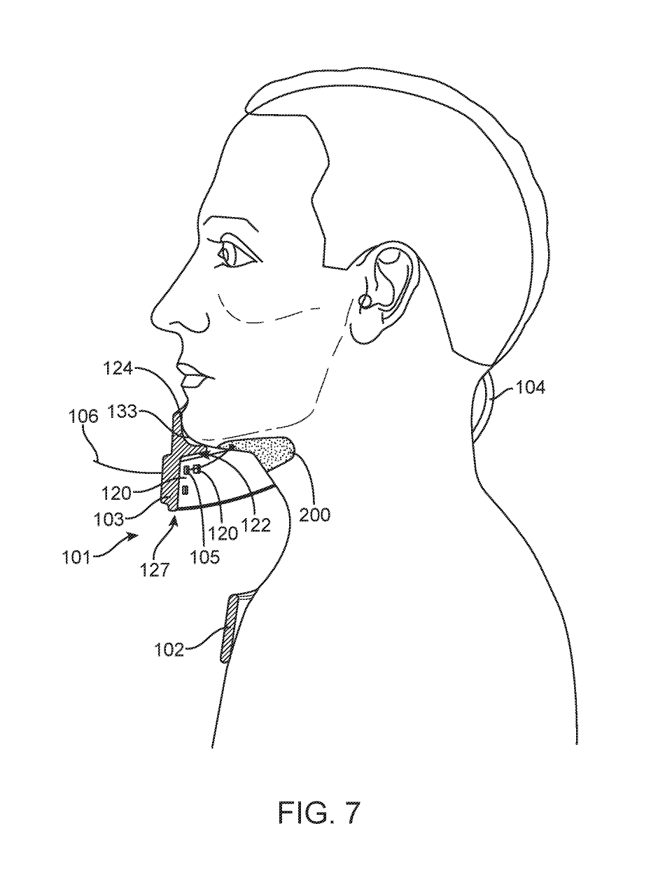

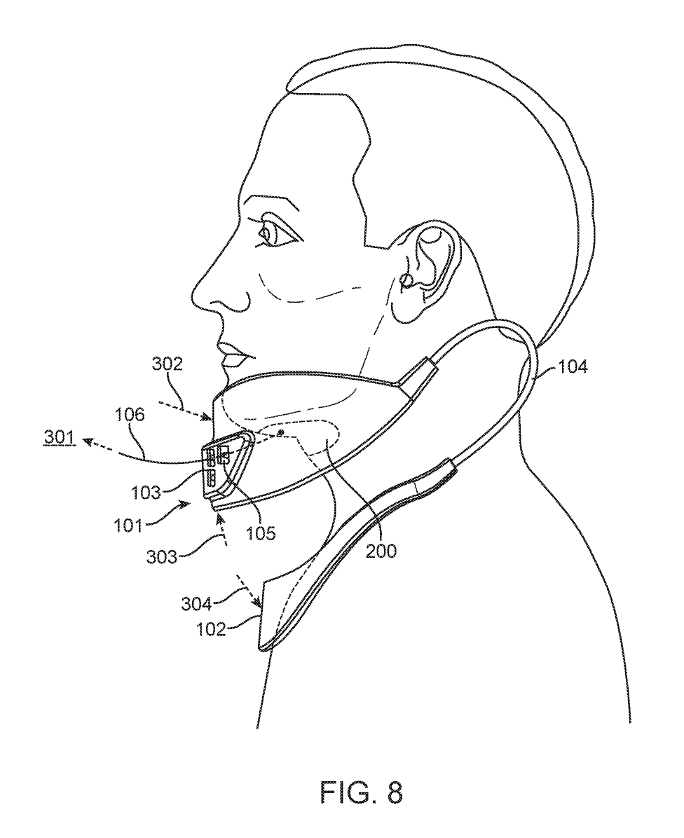

As shown in FIGS. 7 and 8, the attachment member 200 is adhered or otherwise attached to the skin of the patient underneath the jaw and chin of the patient and a tensioning member 106 passes through an aperture 120 of the locking member 105 and is held in tension by the locking member 105 to tension the genioglossus and geniohyoid muscles which maintains the patient's airway and aids in preventing obstructive sleep apnea. In some embodiments, the force is transferred to the hyoid bone to pull the hyoid bone anteriorly to increase the opening of the airway to aid in preventing obstructive sleep apnea.

An example of the forces applied by the neck assembly 100 are illustrated in FIG. 8 which shows the neck assembly 100 fitted to a patient and with the forces indicated by arrows 301, 302, 303, 304, 305. The force indicated by arrow 301 is the pulling or tensioning force applied on the tensioning member 106 and transmitted through the tensioning member and the attachment member 200 to the submandibular tissue and results in tensioning of the genioglossus and geniohyoid muscles which maintains the patient's airway and aids in preventing obstructive sleep apnea. In some embodiments, the force is transferred through the tensioning member and the attachment member 200 to the hyoid bone to pull the hyoid bone anteriorly to increase the opening of the airway.

The force indicated by arrow 302 counteracts the force indicated by arrow 301. In other words, the force indicated by arrow 302 is the reaction force to the force indicated by arrow 301. The force indicated by arrow 302 may be applied in a direction opposite the tensioning force indicated by arrow 301. The force indicated by arrow 301 is also a stabilizing force that stabilizes the collar in an anterio-posterior direction. By balancing the force 301 applied to the tensioning member 106 with the force 302 to the chin or jaw of the patient, airway patency is maintained without moving the jaw or mandible forward.

When worn by the patient, the jaw support 103 and body support 102 may be pressed together, compressing the connecting member 104 such that is applies a force through the jaw support 103 and the shelf 122 towards the patient's chin and jaw, as indicated by the arrow 303. In addition, the connecting member applies a force though the body support 304 onto the chest of the patient, as indicted by the arrow 304. The forces exerted by the connecting member 104, though the jaw support and body support and into the patient, aid in stabilizing the collar 100 between the chin and neck of the patient. By coupling the jaw assembly 103 to the patient via the attachment assembly 200 and by optionally stabilizing the neck assembly by compressing the connecting member 104 such that it applies a force through the jaw support 103 and the shelf 122 towards the patient's chin and jaw, as indicated by the arrow 303, the position of the next assembly 100 is maintained without the use of straps around the top of the head or around the neck.

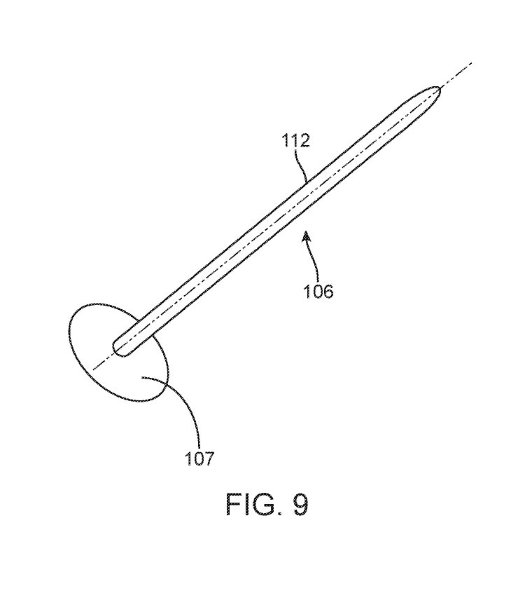

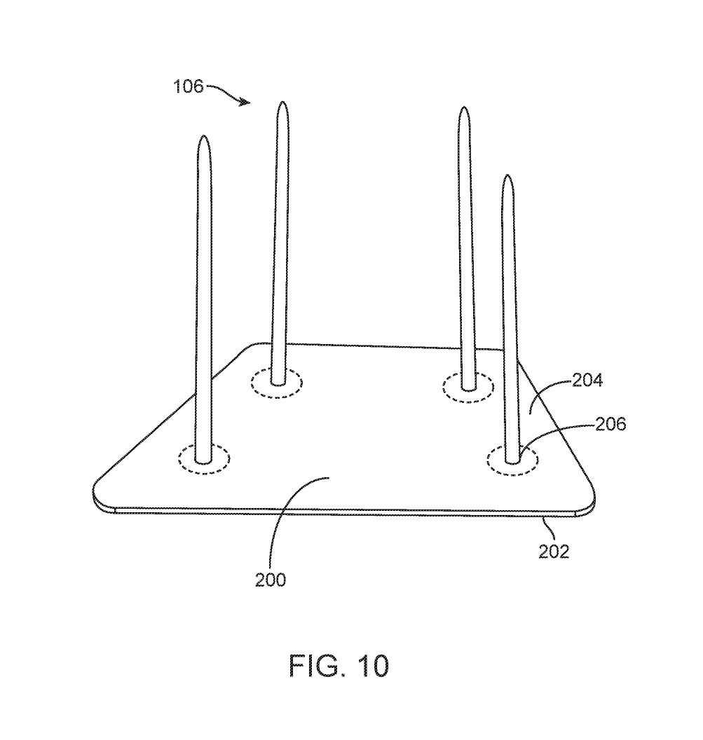

In another embodiment and in accordance to FIG. 9, the tensioning member 106 can be a rigid member having an elongated body, such as a shaft 112 and a flattened base 107 at one end of the shaft 112. The flattened base may be integrally formed with the shaft 112. In some embodiments, the flattened base may be pre-attached to the chin attachment member 200. For example, as shown in FIG. 10.

FIG. 10 depicts a plurality of tensioning members 106 coupled to an attachment member 200. The attachment member 200 may be a sheet having a body facing surface 202 and an external facing surface 204 opposite the body facing surface 202. The attachment member 200 may have a plurality of apertures 206 that are sized and shaped to receive the elongated body of the tensioning member there though, but not the flattened base 107. For example, the diameter or cross sectional shape of the aperture 206 may be substantially similar to the diameter or cross sectional shape of the shaft of the tensioning member 106 such that the shaft may pass though the aperture 206, but the diameter of the aperture 206 may be smaller than the diameter of the flattened base 107 or otherwise shaped such that the aperture 206 resists passage of the flattened base 107 there though.

In some embodiments, the aperture 206 passes though the body facing and external facing surfaces 204 of the attachment member 200, while in some embodiments, such as wherein the attachment member is a multilayered sheet, the aperture may pass though the external facing surface 204 and one or more of the layers, but not though the body facing surface. In such an embodiment, the flattened base 107 may be sandwiched between one or more of the layers.

FIG. 11 shows additional embodiment wherein the tensioning member 106 can have a rigid elongated body pivotally attached to a flattened base 107. In FIG. 11 the flattened base 107 is a sheet material having two arms or extension joined together and extending away from each other in a substantially V-shaped configuration. The tensioning member 106 is coupled to or integrated with the attachment flattened base at the vertex of the arms. The flattened base 107 may be adhered to or otherwise coupled to one or more attachment members 200. In some embodiments, the flattened base 107 is adhered to a patient facing surface of the attachment members 200, while in other embodiments the flatted base 107 may be adhered to an external surface of the attachment member 200, or between layers of the attachment member 200.

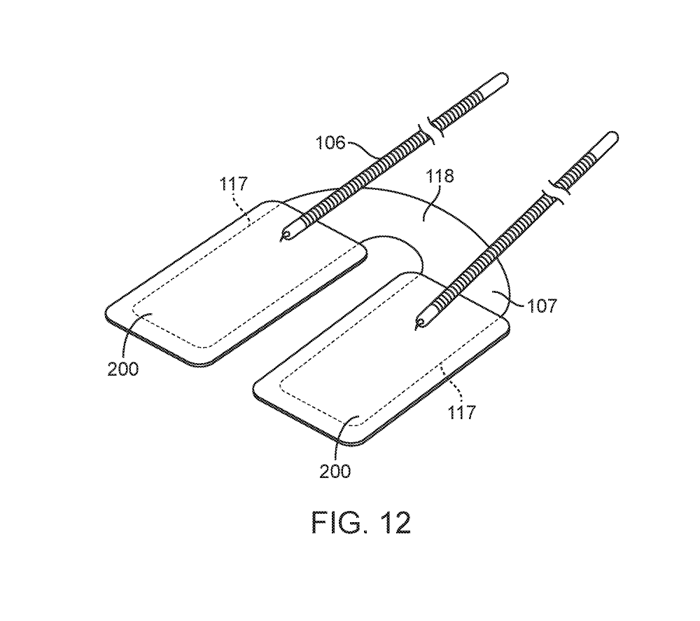

FIG. 12 shows an embodiment wherein the tensioning member 106 can have a rigid elongated body pivotally or rotatably attached to a flattened base 107. In FIG. 12 the flattened base 107 is a sheet material having two arms or extension joined together and extending from each other in a substantially U-shaped configuration wherein the arms extend substantially parallel to each other at connecting portion 118 that extends between the arms. As with the embodiment shown in FIG. 11, the flattened base 107 of FIG. 12 may be adhered to or otherwise coupled to one or more attachment members 200.

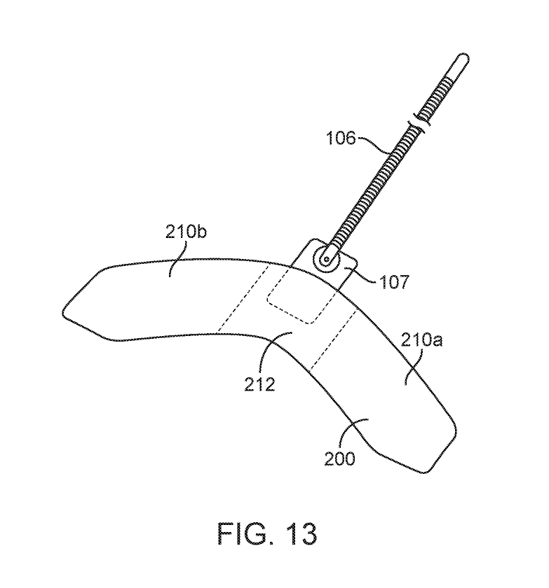

In some embodiments, for example in accordance to FIG. 13, the chin attachment member 200 can have a single tensioning member 106 coupled to a rectangular shaped base 107 that is coupled or otherwise attached to a vertex 212 of two arms 210a, 210b of an attachment an attachment member 200. The two arms 210a, 210b includes first and second ends. The first ends of the arms are joined together or meet at the vertex 212 from which the arms extend and the second ends are terminal ends of the arms at opposite ends of the attachment member 200.

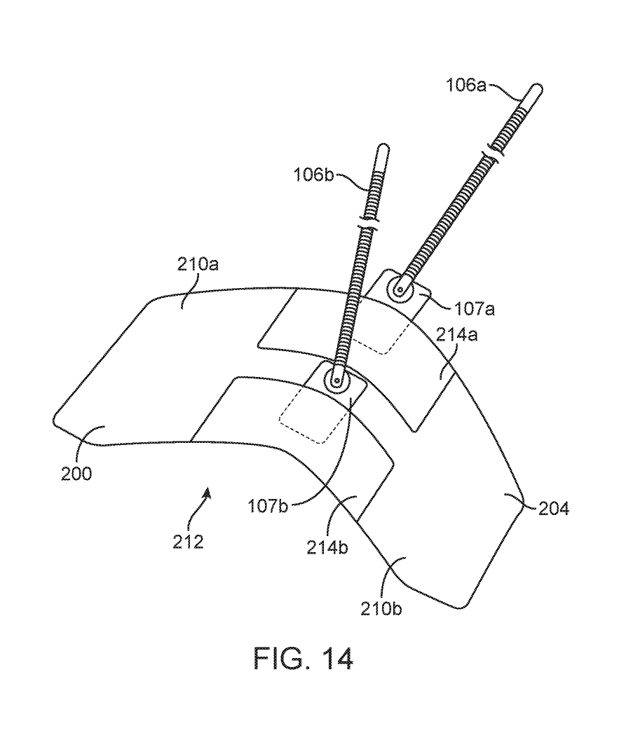

In some embodiments, for example in accordance to FIG. 14, the chin attachment member 200 can have a plurality of tensioning members 106a, 106b coupled to rectangular shaped bases 107a, 107b that are coupled or otherwise attached to a vertex 212 of two arms 210a, 210b of an attachment an attachment member 200. The two arms 210a, 210b includes first and second ends. The first ends of the arms are joined together or meet at the vertex 212 from which the arms extend and the second ends are terminal ends of the arms at opposite ends of the attachment member 200.

As shown in FIG. 14, the bases 107a, 107b are coupled to the exterior facing surface 204 of the attachment member 200. In addition, adhesive sheets 214a, 214b are placed over respective bases 107a, 107b to aid in securing the bases and tensioning member 106a, 106b to the attachment member 200.

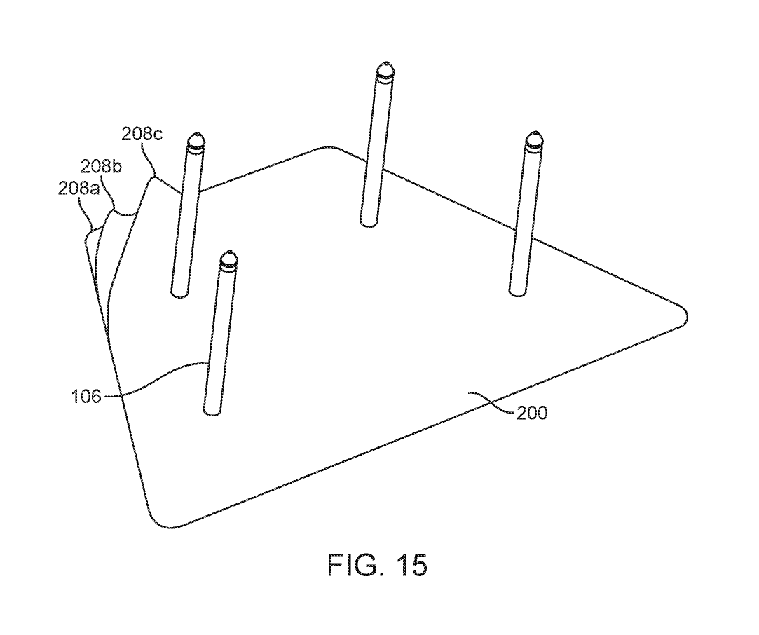

In yet another embodiment and in accordance with FIG. 15, the attachment member 200 can have multiple tensioning members 106 arranged in an array about the body of the attachment member 200 to distribute forces imparted by the tensioning members over a greater area and maintain airway patency during use.

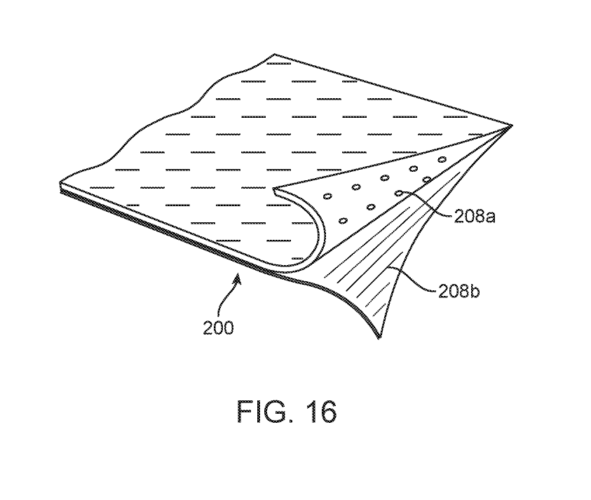

As further illustrated in FIGS. 15 and 16, the body of the attachment member 200 may include one or more sheets 208. Each sheet 208 may be made from the same or different material. For example, she sheets may be made from material including but not limited to thin film polyurethane with adhesive lining, woven or non-woven fabric with adhesive, flexible plastic film or strip with adhesive lining, reinforced fabric with adhesive lining, multi-layer fabrics or films with adhesive lining capable of strong attachment to skin and not cause any irritation to the skin when used for longer duration. For example, sheet 208a may be an adhesive sheet or film and may include the patient facing surface of the attachment member. The middle sheet 208b may be a structural material that provides strength and distributes the forces imparted on the attachment member by the tensioning members 106. The outer layer or external facing layer 208c may also be a structural material. In some embodiments, the external facing layer 208b may have a color that matches the color of a patient's skin.

The attachment member 200 shown in FIG. 16 includes two structural layers 208a, 208b. Each of the structural layers may properties that vary based on the direction of applied forces. For example, the structural layers have include fibers having a first orientation in structural layer 208a and a second orientation in structural layer 208b. In this way, the orientation of the layers 208a, 208b and the fibers therein may be optimized to distribute the forces from the tensioning members 206, through the attachment member 200 and onto the patient.

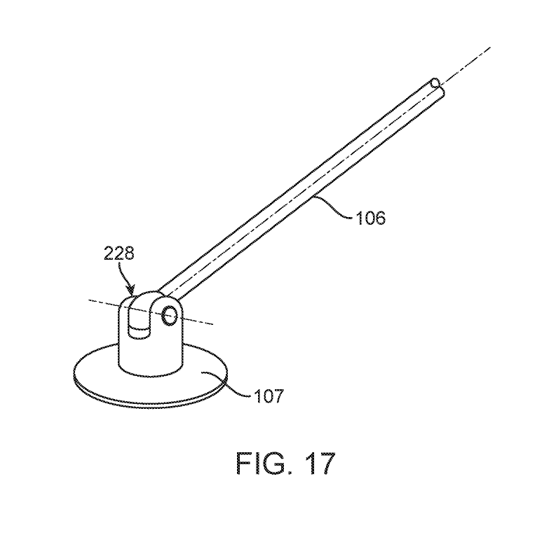

In some embodiments, for example as shown in FIG. 17, the tensioning member 106 can be a rigid member having an elongated body and being pivotably attached to a flattened base 107 via a hinge.

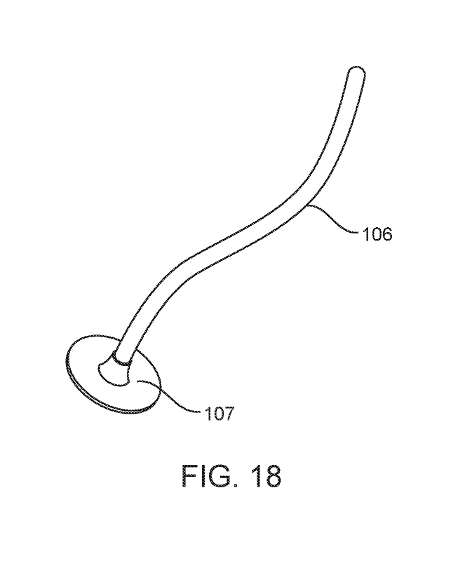

In yet another embodiment and in accordance to FIG. 18, the elongated body of the tensioning member 106 can be in the form of string attached securely to a flattened base 107. In some embodiments, the flattened base is attached to an attachment member 200. In another embodiment, the flattened base 107 can have an adhesive lining that can be used to attach the tensioning member 106 to the patient's body, for example, the user's sub-mandibular area or any other area describe herein, such as the submental, submaxillary, hyoid, subhyoid, and carotid fossa of the face and neck of a patient. In some embodiments, one or more tensioning members 106 may be attached to the patient such that it transfers a force from the tensioning member 106 to the geniohyoid muscle and genioglossus muscle such that the sub-mandibular tissue is moved anteriorly to maintain the tone of the genioglossus and geniohyoid muscles and thereby maintaining airway patency while sleeping.

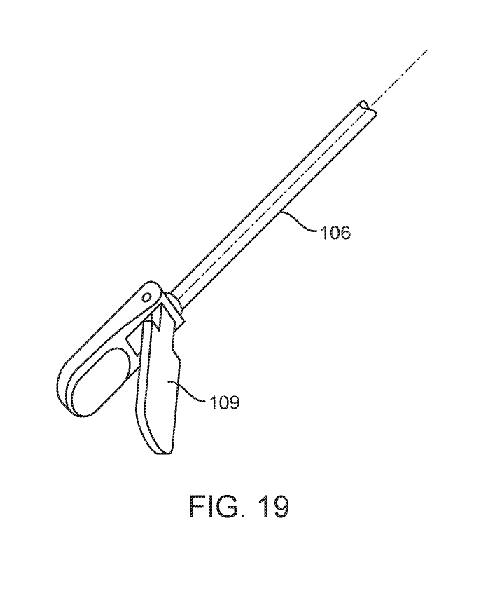

In another embodiment and in accordance to FIG. 19, the tensioning member 106 may be coupled to the attachment member 200 via mechanical clamp 109 adaptable to releasably secure the tensioning member 106 to the attachment member 200.

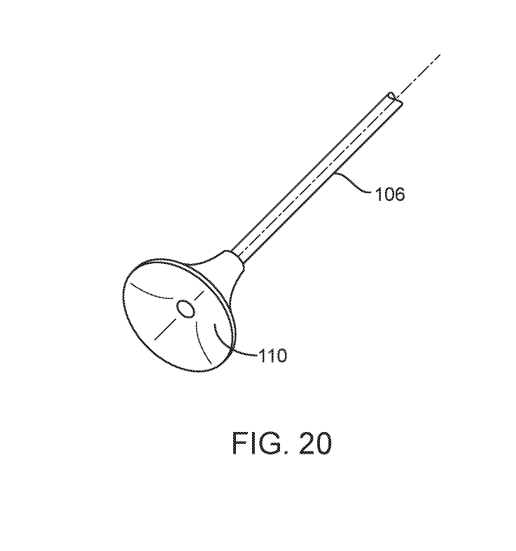

In yet another embodiment and in accordance to FIG. 20, the tensioning member 106 can be coupled to the attachment member via a suction cup 110 at one end of the body of the tensioning member and be releasable coupleable to the attachment member using a vacuum.

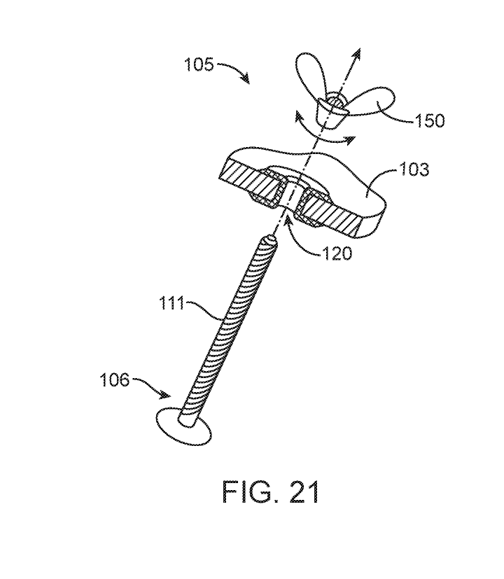

FIG. 21 depicts a cutaway of a jaw support member and locking member 105. The locking member 105 includes a fastener 150 and an aperture 120 though the body of the jaw support 103. The fastener may be a nut, such as the wing nut depicted in in FIG. 21. The wing nut 150 engages with a locking feature on the tensioning member 106. In some embodiments, for example as shown in FIG. 21, the locking feature may be threads 111 along a length of the tensioning member 106.

The locking member 105 is configured to releasably engage the free end of the tensioning member 106 and adapted to apply a tensioning force onto the tensioning member 106 by tightening the fastener, such as the nut 150.

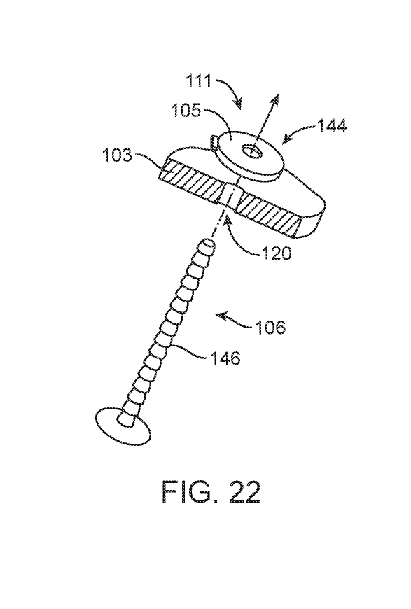

In some embodiments, for example as shown in FIG. 22, the locking member 105 has a locking feature 111 in the form of ratcheting pawl 144 that engages with teeth 146 on the tensioning member 106. In use, the elongated body of the tensioning member 106 passes through the aperture 120 in the jaw support 103 and engages with the ratcheting pawl 144. A tensioning force is applied to the tensioning member and the ratcheting pawl is slid up the elongated body of the tensioning member 106. When the force is released, the ratcheting pawl is pulled against the outward facing surface of the jaw support 103 which maintains its hold on the elongated body of the tensioning member 106 and thereby maintains the tensioned member 106 in tension.

In some embodiments, the fastener 150 may be a threaded nut, friction clamp, lock pin or other type of fastener that to engages the tensioning member 106 and maintains tension of the tension member during use.

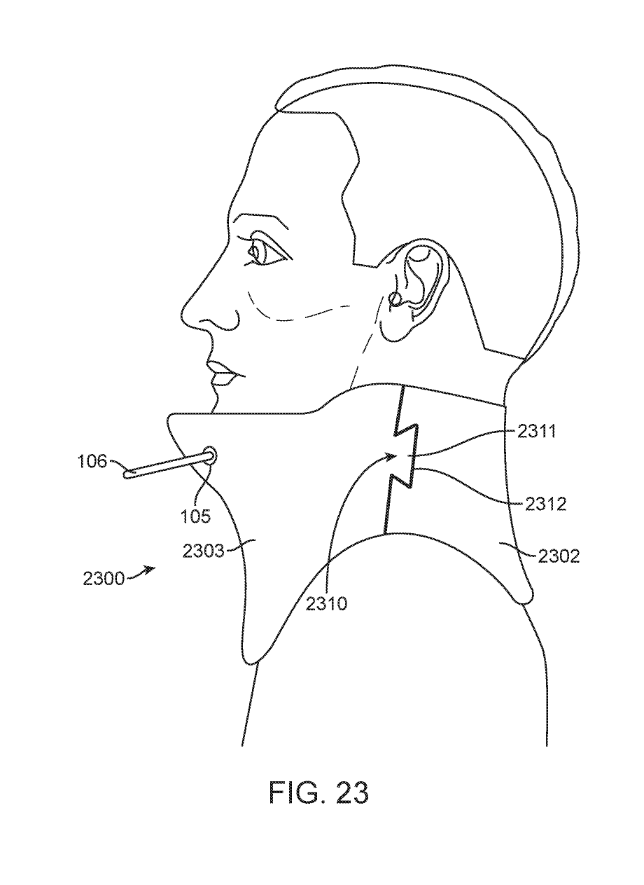

Referring now to FIG. 23 a neck assembly in the form of a two-part neck collar assembly 2300 is shown. The neck collar assembly 2300 includes a front support 2303 and a rear support 2302. The front support 2303 and rear support 2302 may be coupled together via a tongue and groove structure 2310 wherein one of the front support 2303 and the rear support 2302 includes one of the tongue or groove while the other of the supports includes the other of the tongue or groove. For example, the front support 2303 includes a tongue 2311 while the back support 2302 includes the groove 2312. The front and back supports 2303, 2302 encircle or surround the neck of the patient. The front support includes one or more locking members 105 that are configured to engage one or more tensioning members 106 to transmit forces to the underside of a patient's chin and jaw as described herein. The front support 2303 may be a jaw support and include the features of the jaw supports described herein. For example, front support 2303 may include a first, inward or patient facing surface that is configured to face the patient and a second, outward facing surface that faces away from the body of the patient. The patient facing surface may be shaped to correspond to the shape of the outer body of the patient's face, for example the patient's chin and jaw. In some embodiments, the patient facing surface may be configured to comfortably rest on the patient's body at or near one or more of the mental, buccal, parotid, and submaxillary regions of the patient's face.

The front support may also include a shelf that extends from the patient facing surface of the front support. In some embodiments, the shelf extends from a central portion of the front support and may have a shape that corresponds to a shape of the underside of patient's mental or chin region. In some embodiments, the front support may include multiple shelves or discrete shelf portions.

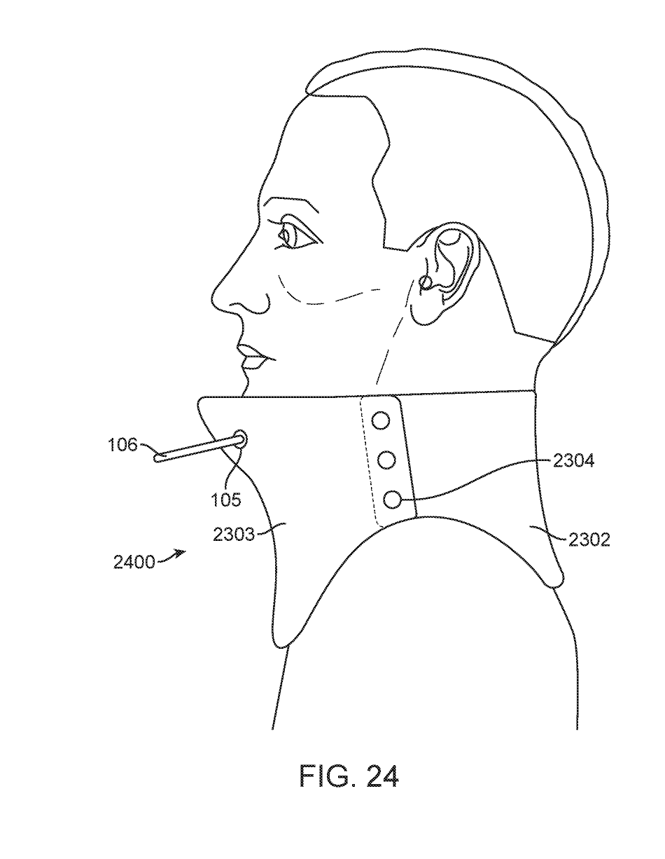

Referring now to FIG. 24 a neck assembly in the form of a two-part neck collar assembly 2400 is shown. Like the neck collar assembly 2300, the neck collar assembly 2400 includes a front support 2303 and a rear support 2302. The front support 2303 and rear support 2302 may be coupled together by one or more snap couplings 2304. The front and back supports 2303, 2302 encircle or surround the neck of the patient. The front support includes one or more locking members 105 that are configured to engage one or more tensioning members 106 to transmit forces to the underside of a patient's chin and jaw as described herein. The front support 2303 may be a jaw support and include the features of the jaw supports described herein. For example, front support 2303 may include a first, inward or patient facing surface that is configured to face the patient and a second, outward facing surface that faces away from the body of the patient. The patient facing surface may be shaped to correspond to the shape of the outer body of the patient's face, for example the patient's chin and jaw. In some embodiments, the patient facing surface may be configured to comfortably rest on the patient's body at or near one or more of the mental, buccal, parotid, and submaxillary regions of the patient's face.

The front support may also include a shelf that extends from the patient facing surface of the front support. In some embodiments, the shelf extends from a central portion of the front support and may have a shape that corresponds to a shape of the underside of patient's mental or chin region. In some embodiments, the front support may include multiple shelves or discrete shelf portions.

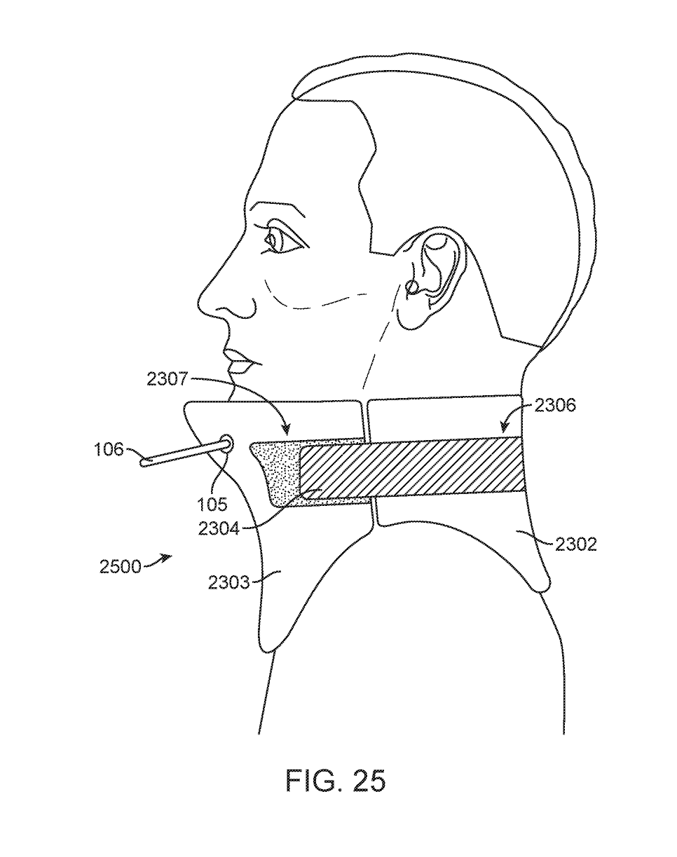

Referring now to FIG. 25 a neck assembly in the form of a two-part neck collar assembly 2500 is shown. Like the neck collar assembly 2300, the neck collar assembly 2500 includes a front support 2303 and a rear support 2302. The front support 2303 and rear support 2302 may be coupled together by a hook and loop coupling 2304. The hook and loop coupling 2304 may include a strap 2306 that includes one of either the hooks and loops of the hook and loop fastener while the other of the hook and loop structure is affixed to either of the front or back supports. As shown in FIG. 25, the other of the hook and loop structure 2308 is affixed to the front support 2303.

The front and back supports 2303, 2302 encircle or surround the neck of the patient. The front support includes one or more locking members 105 that are configured to engage one or more tensioning members 106 to transmit forces to the underside of a patient's chin and jaw as described herein. The front support 2303 may be a jaw support and include the features of the jaw supports described herein. For example, front support 2303 may include a first, inward or patient facing surface that is configured to face the patient and a second, outward facing surface that faces away from the body of the patient. The patient facing surface may be shaped to correspond to the shape of the outer body of the patient's face, for example the patient's chin and jaw. In some embodiments, the patient facing surface may be configured to comfortably rest on the patient's body at or near one or more of the mental, buccal, parotid, and submaxillary regions of the patient's face.

The front support may also include a shelf that extends from the patient facing surface of the front support. In some embodiments, the shelf extends from a central portion of the front support and may have a shape that corresponds to a shape of the underside of patient's mental or chin region. In some embodiments, the front support may include multiple shelves or discrete shelf portions.

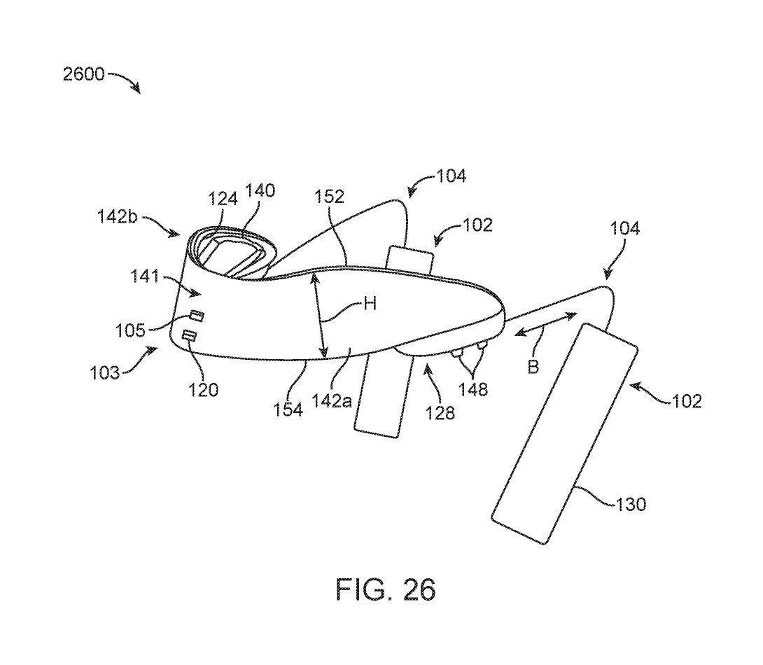

FIG. 26 depicts a simplified embodiment of a neck assembly 2600 that includes a jaw assembly 103 and body supports 102. As discussed above, with respect to other embodiments herein, the jaw support 103 is coupled to the connecting member 104 via a coupling 128. The jaw support 103 may be configured to engage with the jaw and a surface of the underside of a patient's chin to apply an anterior force to the skin under the jaw. This force is transferred though the submandibular tissue and results in tensioning of the genioglossus and geniohyoid muscles which maintains the patient's airway and aids in preventing obstructive sleep apnea. In some embodiments, the force is transferred to the hyoid bone to pull the hyoid bone anteriorly to increase the opening of the airway. This aspect may be useful to prevent more severe obstructive sleep apnea.

The jaw support 103 may be a curved structure having a central portion 141, also called the mental portion that is shaped to fit with a patient's chin, the mental region of the patient's face. Two arms 142a, 142b extend from the central portion 141 of the jaw support. The two arms 142a, 142b are sharped to fit with one or more of the buccal, parotid, and submaxillary portions of a patient's face. The central portion 141, and the two arms 142a, 142b may form a substantially U-shaped structure or semicircular structure that fits the shape of the patient's chin and jaw.

The body of the jaw support 103 includes a first, inward or patient facing surface 124 that is configured to face the patient and a second, outward facing surface 125 that faces away from the body of the patient. Like the patient facing surface 130 of the body supports 102, the patient facing surface 124 may be shaped to correspond to the shape of the outer body of the patient's face, for example the patient's chin and jaw. In some embodiments, the patient facing surface 124 may be configured to comfortably rest on the patient's body at or near one or more of the mental, buccal, parotid, and submaxillary regions of the patient's face.

The jaw support 103 of the neck assembly 2600 may include a shelf 122, such as a shelf depicted in FIGS. 1, 3, and 4. The shelf may extend from the patient facing surface 124 of the jaw support 103. In some embodiments, the shelf 122 extends from a central portion 141 of the jaw support 103 and may have a shape that corresponds to a shape of the underside of patient's mental or chin region. In some embodiments, the shelf 122 extends from the one or more of the arms 142a, 142b and may have a shape that corresponds to a shape of the underside of a patient's jaw, in particular the submaxillary region of the patient's face.

In some embodiments, the shelf may extend from the patient facing surface 124 from the first of the arms 142a, through the central portion of the 141, and the second of the arms 142b. In some embodiments, the jaw support may include multiple shelves 122 or discrete shelf portions. For example, the jaw support may include a first shelf that extends from the inner surface 124 at the central portion 141 of the jaw support 103, and second and third shelves that extend from the inner surface 124 of each of the arms 142a, 142b.

The jaw support 103 may include a plurality of couplings, such as locking members 105, to allow for passage and retention of a tensioning member 106. The locking member 105 may include an aperture 120 through the body of the jaw support 103. The aperture 120 allows for passage of the tensioning member 106 through the jaw support 103 and permits a user, such as a patient, to pull on and tension the tensioning member 106. The locking member 105 may also include a means for retaining the tensioning member 106 (see e.g., FIGS. 5-7). For example, a locking member 105 may include a pawl 121 within or proximate to the aperture that engages with an adjustment feature, such as teeth on the tensioning member 106, to form a ratchet coupling that allows movement of the tensioning member 106 though the locking member 105 and aperture 120 in a first direction and resists movement of the tensioning member 106 in a second direction, which may be opposite the first direction. Such embodiments may allow the user to tension the tensioning member 106 by pulling on it in a first direction and then maintain tension in the tensioning member 106 when the pawl 121 engages with teeth of the tensioning member, see for example, the teeth 146 of the tensioning member 106 in FIG. 11. In some embodiments, the adjustment feature can be in the form of a ratchet, threaded screw, rough surface for friction locking, adjustment holes or other arrangement.

As with other embodiments described herein, the jaw assembly 103 may be coupled to a chin attachment member 200 that is applied under the chin of the user using adhesive, as described above, vacuum suction, or mechanical clamping. Preferably, the chin attachment member 200 is adhesively applied under the chin of the user.

In some embodiments, the jaw support 103 may include a plurality of couplings, such as locking members 106, arranged in an array on the central portion 141 of the jaw support 103. In some embodiments, the locking members 106 may be aligned with a centerline of the jaw support 103 or otherwise, as described herein.

The height H of the jaw assembly 103 may extend from an upper edge 152 to a lower edge 154. Unlike the embodiment in FIG. 1, where the lower edge of the jaw support 103 extends substantially below the submaximal area or jaw bone of the patient when worn by the patient, the lower edge 154 of the jaw assembly 103 shown in FIG. 25 terminates proximate the submaximal area or jaw bone of the patient. For example, the lower edge 154 may terminate less than 0.5 cm, less than 1 cm, or less than 1.5 cm below the submaximal area or jaw bone of the patient.

As shown in FIG. 26, the jaw assembly may include a pad 140 attached to the patient facing surface of the jaw assembly 130.

The connecting members 104 and body supports 102 are optional. In some embodiments, the jaw assembly is supported and attached to the patient by the tensioning members 106 and attachment members 200 without the use of the connecting members 104 and the body supports 102, such that the jaw assembly with the tensioning members and attachment assembly are self-supporting.

The neck assembly 2600 of FIG. 26 includes couplings 128 that slidingly couple with the connecting members 104 such that the distance between the jaw assembly 103 and the body supports 102 may be adjusted by sliding the connecting members 104 into or out of the couplings 128. When the connecting members and body supports are at the desired distance, fasteners 148, which may be a set screw, engage with the connecting member and lock it in place.

The body supports 102 depicted in FIG. 26 may be elongated members that, rather than engaging with the chest of a patient, are shaped to engage with the neck of a patient. As shown in FIG. 26, the body supports may be substantially rectangular in shape with a length to width aspect ratio of between 3:1 and 6:1. When worn by the patient, the body supports engage with the neck in an orientation that extends in a direction from the head towards the chest of the patient and along either side of the larynx of the patient.

### Method and Example of Use

With reference to the FIGS. 5-8, the assembly 100 is applied to the neck and head. The assembly 100 is adjusted for comfortable fit and is placed about or around the neck.

The body support 102 and jaw support 103 are then further adjusted for optimal position and comfort. The chin attachment member 200 is adhesively applied under the chin. The tensioning members are engaged with the locking members of the jaw assembly and are pulled in the anterior direction to the tissue of the sub-mandibular region. This attachment assembly transmits the force of the tensioning member 106 to the underlying geniohyoid muscle and genioglossus muscle and results in the movement of the sub-mandibular tissue anteriorly to maintain the tone of the genioglossus and geniohyoid muscles and thereby maintaining airway patency while sleeping or during application or use of anesthesia.

In another method, the chin attachment member 200 provides a force at a location anterior to the hyoid bone either optionally in conjunction to the force applied at sub-mandibular region or without the force applied at the sub-mandibular region. This additional force is useful to mechanically pull the hyoid bone anteriorly and increase the opening of the airway in patients with more severe obstructive sleep apnea.

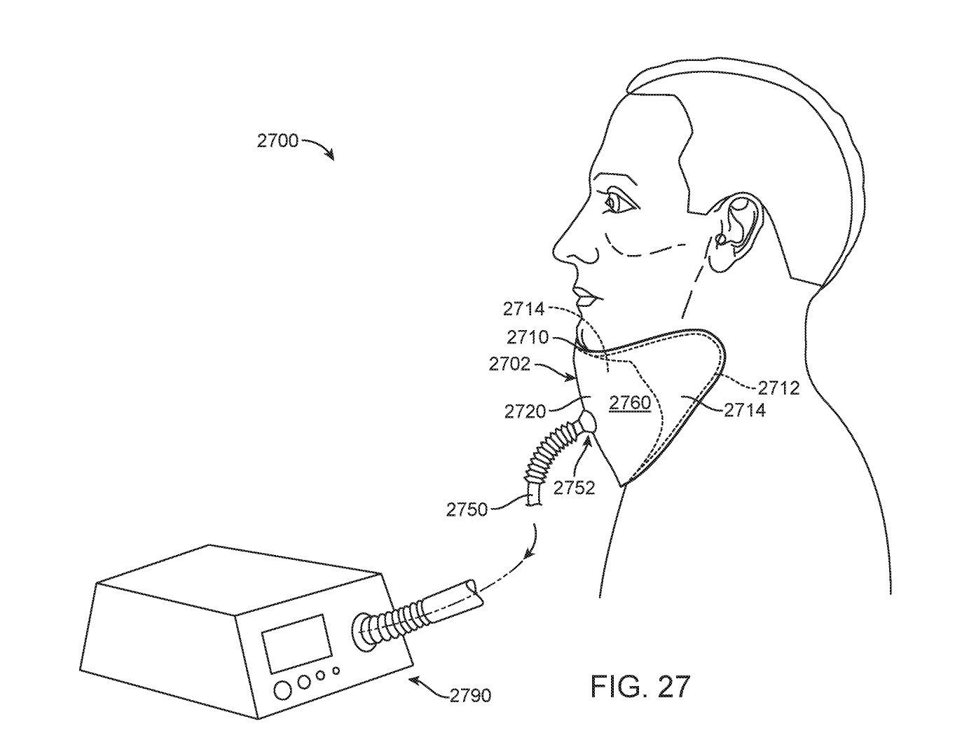

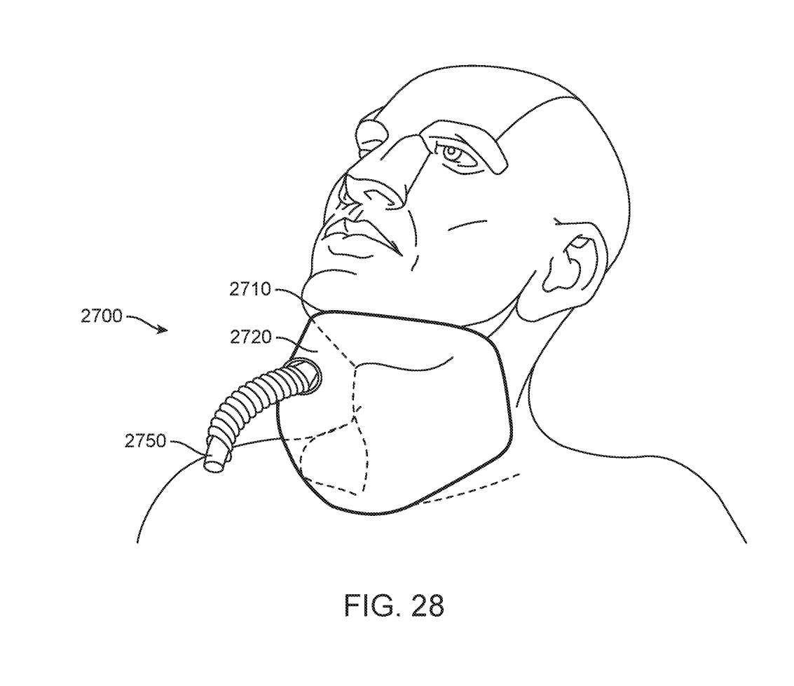

FIGS. 27 and 28 illustrate a suction system 2700 according to one or more embodiments disclosed herein. The suction system 2700 includes a suction collar 2702 connected via a hose 2750 to a suction device 2790. The suction collar 2702 may be placed against a s throat and chest area, as shown in FIG. 27. The suction collar 2702 includes a flexible airtight seal 2712 along a patient mating edge of a rigid dome 2720 of the suction collar 2702. The patient mating edge 2710 may further includes a flexible seal 2712. The flexible seal 2712 may be made from materials such as, but not limited, to polymer foam, silicone, rubber, Polyurethane, and other flexible materials capable of forming a seal between the patient mating edge 2710 and the patient. In some embodiments, the flexible seal may be or include an adhesive lining that adheres the suction collar 2702 to the patient.

The seal formed between the suction collar 2702 and the patient is preferably air-tight or substantially air-tight. Such an air-tight seal minimizes air leakage and therefore minimizes the operation of the suction device 2790 in maintaining the vacuum or negative pressure within the cavity 2760 formed between the patient and the suction collar 2702.

The patient mating edge 2710 and the rigid dome 2720 may be sized and shaped to fit an individual patient's anatomy or may be of a generic size and shape such that it may fit many patients. The rigid dome of the suction collar 2702 includes a patient facing concavity and is shaped to extend from an upper edge of the patient mating edge 2710 that is shaped to mate with a jaw of the patient, to a lower edge of the patient mating edge 2710 that is shaped to mate with the chest of a patient. The body of the rigid dome 2720 includes two lateral extensions 2714 that extend along lateral sides of the patient along the jaw line or jaw bone and the chest of the patient to the sides of the neck of the patient.

When applied to the neck and throat area of a patient, a cavity if formed between the suction collar 2702 and the patient's jaw, neck, and chest. This cavity is coupled in fluid communication with a suction device 2790, which may be a vacuum pump.

When suction is applied by the suction system 2700 by the suction device 2790 a negative pressure within the cavity 2760. The negative pressure is negative relative to the environment outside the cavity 2760. This negative pressure acts on the space between the rigid dome and flexible airtight seal and in turn on the user's throat area to create an anterior pull on the user's sub-mandibular tissue to maintain the tone of the genioglossus and geniohyoid muscles and thereby maintaining airway patency while sleeping or to otherwise open up the airway and aid in reducing the occurrence of sleep apnea.

The suction device 2790 may apply a continuous suction or intermittent suction. Intermittent suction may allow greater intermittent suction and also aid in preventing unwanted injury to the patient's skin as compared to continuous suction.

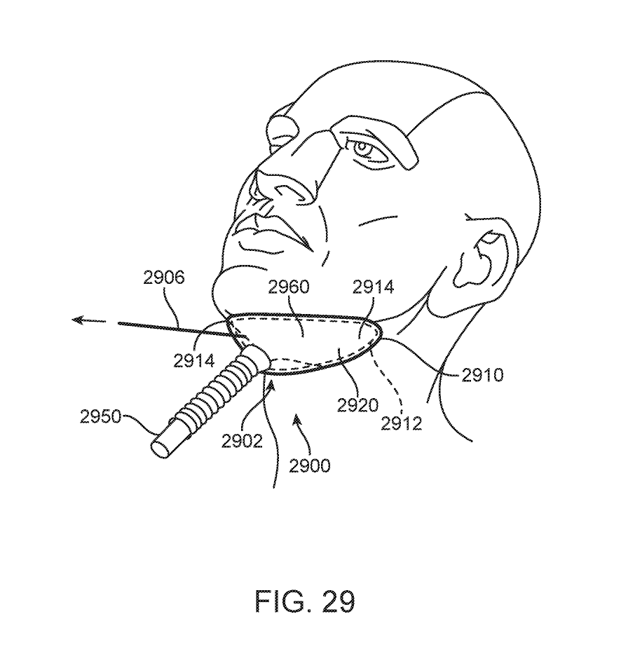

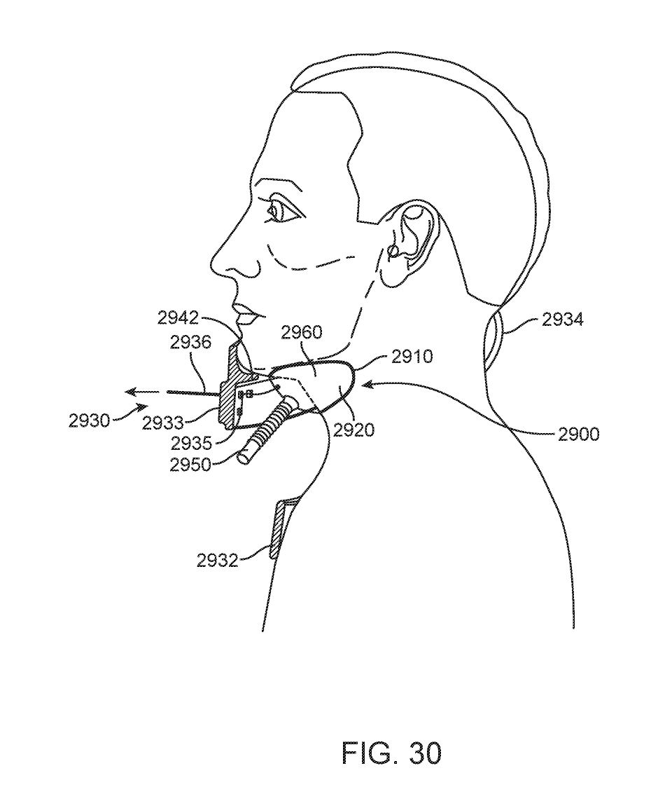

FIGS. 29 and 30 show a suction assembly 2900 used in conjunction with a neck collar assembly 2930. FIG. 29 shows the suction assembly 2900 attached to a patient without the neck collar assembly 2930. In some embodiments, the suction assembly may be used without the neck collar assembly 2930.

Like the assembly 2720 of the suction system 2700, the suction assembly 2900 includes a suction collar 2902 connected via a hose 2950 to a suction device, not shown. The suction collar 2902 is sized and shaped to be placed against a user's sub-mandible area, as shown in FIG. 30. The suction collar 2902 may include a flexible airtight seal 2912 along a patient mating edge 2910 of a rigid dome 2720 of the suction collar 2902. The flexible seal 2912 may be made from materials such as, but not limited, to polymer foam, silicone, rubber, Polyurethane, and other flexible materials capable of forming a seal between the patient mating edge 2910 and the patient. In some embodiments, the flexible seal may be or include an adhesive lining that adheres the suction collar 2920 to the patient.

The seal formed between the suction collar 2902 and the patient is preferably air-tight or substantially air-tight. Such an air-tight seal minimizes air leakage and therefore minimizes the operation of the vacuum pump 2990 in maintaining the vacuum or negative pressure within the cavity 2960 formed between the patient and the suction collar 2920.

The patient mating edge 2910 and the rigid dome 2920 may be sized and shaped to fit an individual patient's anatomy or may be of a generic size and shape such that it may fit many patients. The rigid dome of the suction collar 2902 includes a patient facing concavity and is shaped to fit the sub-mandibular area of a patient. The body of the rigid dome 2920 includes two lateral extensions 2914 that extend along lateral sides of the patient along the jaw line or jaw bone and the chest of the patient to the sides of the neck of the patient.

When applied to the neck and throat area of a patient, a cavity if formed between the suction collar 2902 and the patient's sub-mandibular area. This cavity is coupled in fluid communication with a suction device, not shown, which may be a vacuum pump.

When suction is applied to the cavity 2960 by the vacuum pump, a negative pressure is built up within the cavity 2960. The negative pressure is negative relative to the environment outside the cavity 2960. This negative pressure acts on the space between the rigid dome and flexible airtight seal and in turn on the user's throat area to aid in coupling the suction collar 2902 to the patient and to create an anterior pull on the user's sub-mandibular tissue to maintain the tone of the genioglossus and geniohyoid muscles and thereby maintaining airway patency while sleeping or to otherwise open up the airway and aid in reducing the occurrence of sleep apnea.

In FIG. 29, the suction assembly 2900 is used in conjunction with a neck collar assembly 2930. The neck collar assembly 2930 may be substantially similar to the neck collar assemblies discussed elsewhere herein. For example the neck collar assembly 2930 may be similar to or the same as the neck collar assembly 100, shown in FIG. 1, or the neck collar assembly 2900, shown in FIG. 26.

As with the neck assembly 100, the neck assembly 2930 may include a body support 2932 connected to a jaw support 2933 via a connecting member 2934. The body support 2932, jaw support 2933, and connecting member 2934 may be similar to the corresponding body support 102, jaw support 103, and connecting members 104 of the neck assembly 100 or neck assembly 2600 and include features such as locking members 2935, shelf 2942, and other features and elements of the neck assembly 100 or next assembly 2600.

In the embodiment shown in FIG. 29 the suction assembly 2900, in addition to providing a negative pressure to the sub-mandibular area of the patient, may also be an attachment member, such that is acts to transmit a force from the neck assembly 2930 to the patient. A force transmitting means, such as a tensioning member 2936 couples the neck assembly 2930 to the suction assembly 2900. As shown, the suction assembly 2900 may be configured to be attached to the underside of the chin and jaw of a patient via a suction or negative pressure formed within the chamber 2960. The suction assembly 2900 may be sized and shaped to be coupled to the patient at one or more of the submental, submaxillary, hyoid, subhyoid, and carotid fossa of the face and neck of a patient. In some embodiments, the suction assembly 2900 may be sized and shaped such that it transfers a force from the tensioning member 2936 to the geniohyoid muscle and genioglossus muscle such that the sub-mandibular tissue is moved anteriorly to maintain the tone of the genioglossus and geniohyoid muscles and thereby maintaining airway patency while sleeping or during administration of anesthesia, in much the same way as the attachment members do, as discussed above.

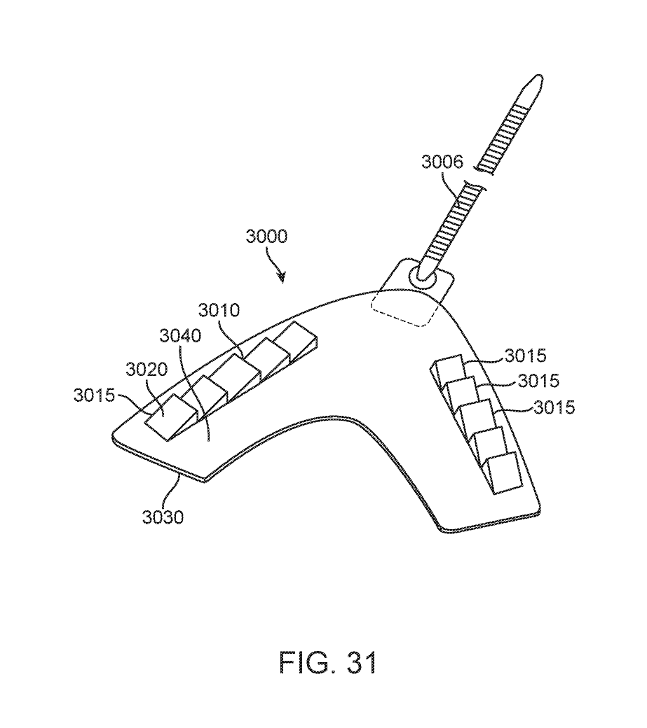

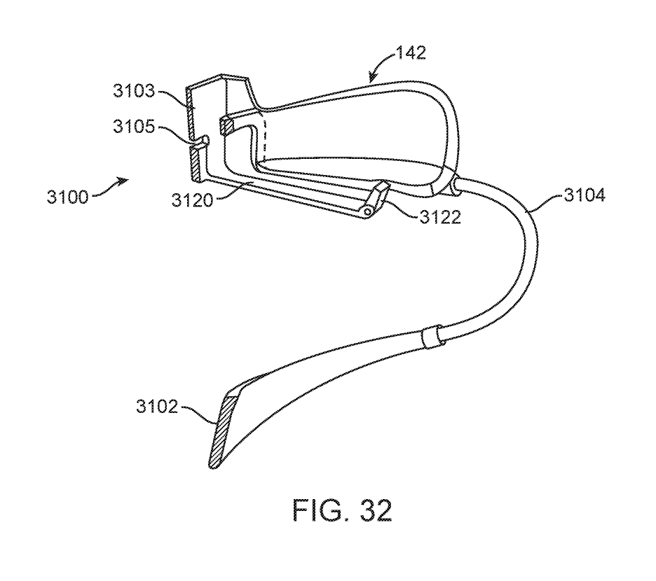

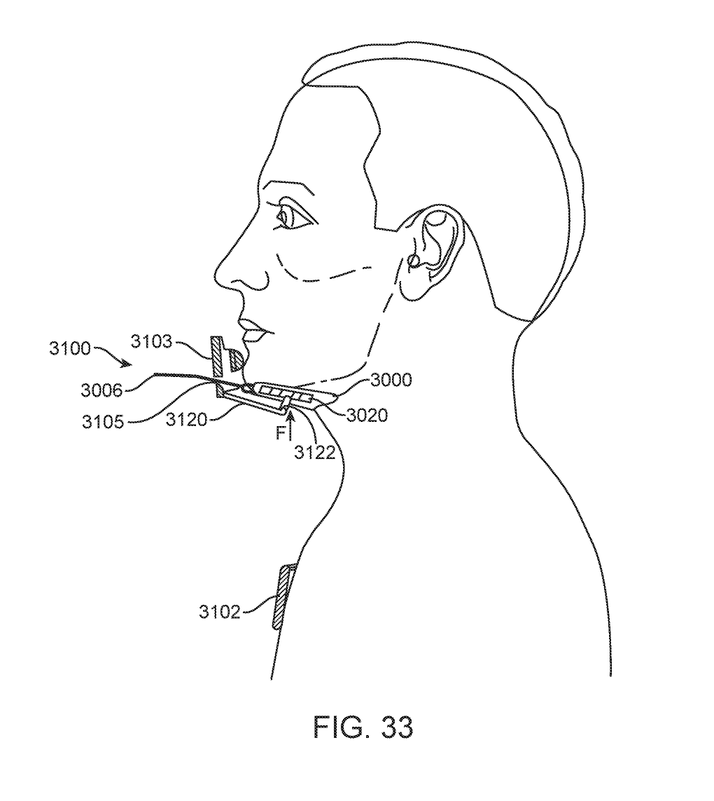

FIG. 31 shows an attachment member 3000. Similar to other attachment members, the attachment member 3000 may include an adhesive on the patient facing surface 3030 that adheres the attachment member 3000 to a submandibular area of a patient. The attachment member 3000 also includes a tensioning member 3006 that may engage with a locking member 3105 of a neck assembly, see, for example, FIGS. 32 and 33. The tensioning member transfers force from a neck assembly to the attachment member 3000 which is then transferred to the patient, such as described above.

In addition, the attachment member 3000 may also include teeth 3015 on an exterior surface of the attachment member 3000. The teeth 3015 may be asymmetrical such that they include a low or moderately sloped surface 3020 and a steep surface 3010. The slop of the low or moderately sloped surface 3020 is such that a pawl 3122 (see FIGS. 32 and 33) slides over the low to moderately sloped surface 3020. The steep surface 3010 has a steep slope such that the steep surface 3010 engages the pawl and resists disengagement. In this way, the teeth 3015 and the pawl 3122 act as a ratchet to allow movement of the neck support in one direction, such as towards the patient's body, but resists movement in another direction, such as an opposite direction, for example, away from the patient's body.

The attachment member 3000 may be used in with a neck support, such as the neck support 3100 shown in FIG. 32. This neck support includes features similar to the features shown in the neck support 100, neck support 2600, or next support 2930. The neck support 3100 includes a jaw assembly 3103 that includes a locking member 3105 and arms 142. The jaw assembly 3103 is coupled to the body support 3102 via the connecting member 3104.

The Jaw assembly 3103 of the neck support 2600 includes a beam 3120 that extends from body of the jaw assembly 3103. The beam 3120 may be rigid or flexible and includes a pawl 3122 at a distal end of the beam. The pawl may be a rotary lever that is rotatably coupled to the beam. In some embodiments, the pawl is fixedly coupled or integrated into the distal end of the beam 3120.

Referring now to FIG. 33, the attachment member 3000 and neck assembly 3100 are shown in use on a patient. As shown, the attachment member is adhered to the patient in a sub-mandibular area. The neck assembly 3100 is coupled to the attachment member 3000 via a tensioning member 3006. An anterior force is applied to the patient's sub-mandibular area via the attachment member 3000, the tensioning member 3006 and the neck assembly 3100 to maintain airway patency, as described above. In some embodiments, the adhesive that couples the attachment member 3000 to the patient may not be strong enough to couple the attachment member for long periods of time or to maintain the coupling when high forces are applied to it by the tensioning member. Therefore, the mean 3120 and pawl 3122 may exert a force F onto the attachment member 3000 to assist in coupling the attachment member to the patient. The force may be a in direction that includes a component that is perpendicular to the surface of the attachment member 3000, such that the beam 3120 and pawl 3122 push the attachment member towards the patient.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

While the preferred embodiments of the invention have been described, it should be understood that various changes, adaptations and modifications may be made therein by those skilled in the art without departing from the spirit of the invention and the scope of the appended claims.

1. A device for aiding in preventing of obstructive sleep apnea, the device comprising:
a chin attachment member including a coupling to couple to an underside of a chin of a patient;
a jaw support shaped to engage a jaw of the patient and having at least one locking member; and
a body support shaped to rest on a chest of the patient.

2. The device as claimed in claim 1, wherein the coupling is an adhesive surface of the chin attachment member.

3. The device as claimed in claim 1, further comprising a connection member, the connection member coupling the jaw support to the body support.

4. The device as claimed in claim 3, wherein the connection member is a spring.

5. The device as claimed in claim 3, wherein the connection member is rigid.

6. The device as claimed in claim 1, further comprising a tensioning member having an elongated body extending between a first end and a second end, the first end configured to couple to the chin attachment member, and
wherein the at least one locking member is configured to receive the second end of the tensioning member.

7. The device as claimed in claims 1, wherein the locking member is configured to engage the tensioning member.

8. The device as claimed in claim 7, wherein the jaw support and locking member are configured to hold the tension member in tension with the attachment member and apply a force the anterior direction to the tissue of the sub-mandibular region of a patient.

9. The device as claimed in claim 8, wherein the tensioning member comprises an adjustment feature in the form selected from teeth, threads, a rough surface for friction locking, and adjustment apertures.

10. The device as claimed in claim 6, wherein the elongated body of the tensioning comprises polyamide, polypropylene, polycarbonate, ABS, nylon, polyester, or silk.

11. The device as claimed in claim 8, wherein the jaw support and locking member are configured to hold the tension member in tension without protraction of a mandible of the patient.

12. A method for treating obstructive sleep apnea, the method comprising:
attaching an attachment member to a patient at a location under the chin of the patient;
coupling a jaw assembly to the patient, the jaw assembly being coupled such that it is held about the mental and buccal portions of the patient's face; and
applying a force the tissue of the sub-mandibular region of the patient in an anterior direction.

13. The method of claim 12, further comprising:
maintaining airway patency while the patient is sleeping or under treatment of anesthesia.

14. The method of claim 12, further comprising:
coupling the attachment member to the jaw assembly with a tensioning member.

15. The method of claim 14, wherein the tensioning member is an elongated body having a first end and a second end, and being coupled to the attachment member at a first end being received though a locking member of the jaw assembly.

16. The method of claim 15, further comprising:
engaging the elongated body of the tensioning member by the locking member to hold the tensioning member in tension between the locking member and the attachment member.

17. The method of claim 16, wherein the tensioning member transmits the force from the jaw assembly though the attachment member to the tissue of the sub-mandibular region of the patient.

18. The method of claim 12, wherein airway patency is maintained without protraction of a mandible of the patient.

19. The method of claim 12, further comprising:
providing a body assembly coupled to the jaw assembly; and
placing the body support on the upper chest of the patient.

20. The method of claim 12, further comprising:
providing a body assembly coupled to the jaw assembly via connecting members slidingly engaged with the jaw assembly at a coupling; and
adjusting the body assembly position relative to the jaw assembly by sliding the connecting members in the coupling.
